(19) United States
(12) Patent Application Publication
(10) Pub. No.: US 2015/0283093 A1
(43) Pub. Date: Oct. 8, 2015

(54) NANOENCAPSULATION OF CURCUMINOID METABOLITES - PROCESS, COMPOSITION AND APPLICATIONS THEREOF

(71) Applicants: Muhammed Majeed, Edison, NJ (US); Kalyanam Nagabhushanam, East Windsor, NJ (US)

(72) Inventors: Muhammed Majeed, Edison, NJ (US); Kalyanam Nagabhushanam, East Windsor, NJ (US)

(21) Appl. No.: 14/680,076

(22) Filed: Apr. 7, 2015

Related U.S. Application Data
(60) Provisional application No. 61/975,913, filed on Apr. 7, 2014.

(51) Int. Cl. A61K 9/51; A61K 31/12

(52) U.S. Cl. CPC A61K 9/5138; A61K 31/12; A61K 9/5192

(57) ABSTRACT

Disclosed is a process wherein nano-sized particles containing curcumin metabolites are prepared by the rapid expansion of subcritical solutions of curcumin metabolites and a polymeric substance into liquid solvents precipitating the nano-sized particles.

This invention is non-provisional filing of U.S. Provisional Patent Application No. 61/975,913 filed on 7 Apr. 2014.

### BACKGROUND OF THE INVENTION

1. Field of the Invention

The present invention in general pertains to novel delivery systems for natural metabolites. More specifically, the present invention pertains to tetrahydrocurcumin loaded and trapped onto biologically acceptable polymeric nanoparticles.

2. Description of Prior Art

Encapsulation is an alternative technique to improve the stability nano-sized particles of natural molecules of biological importance. However, the conventional techniques like ionotropic gelation, liposomes, emulsification, spray-drying, and freeze-drying suffer from technical disadvantages like (a) the generation of particles with broad size distributions; (b) lack of control over particle size; (c) long processing time; (d) use of excessive organic solvents that need to be removed from the final product; and (e) the requirement of large quantities of surfactants. To overcome these limitations, supercritical fluid technology has been investigated to produce fine polymeric particles loaded with active compounds, in the instant invention tetrahydrocurcumin, tetrahydrodemethoxycurcumin, tetrahydrobisdemethoxycurcumin, hexahydrocurcumin, octahydrocurcumin and other metabolites of curcuminoids.

Accordingly, it is the principle objective of the present invention to outline a process where the rapid expansion of subcritical solutions into liquid solvents was used as a single-step promising encapsulation technology for curcuminoid metabolites, in particular tetrahydrocurcumin.

The present invention fulfils the aforesaid objective and provided further related advantages.

### SUMMARY OF THE INVENTION

The invention relates to the production of tetrahydrocurcumin loaded and entrapped onto biologically acceptable polymeric nanoparticles. Rapid expansion of subcritical solutions into liquid solvents was used as a single-step promising encapsulation technology to overcome the limitations of conventional techniques.

### DESCRIPTION OF THE MOST PREFERRED EMBODIMENT

In the most preferred embodiment, the present invention relates to a process of nanoencapsulating curcuminoid metabolites like tetrahydrocurcumin, said process comprising steps of
a) Expanding a solution containing tetrahydrocurcumin (THC) 2% and poly(L-lactide) 2% in a mixture of ethanol and carbon dioxide (3:2 wt/wt) across a nozzle (50 μm diameter and L/D=4) into water with a pre-established constant pre-expansion temperature (Tpre) of 55-100 deg C and pre-expansion pressure (Ppre) of 210-330 bar;
b) Collecting the precipitates (nanoencapsulated tetrahydrocurcumin) as a suspension at about 10-15 cm from the tip of the nozzle; and
c) Filtering the suspension obtained in step b through vacuum suction through a nylon membrane (0.45 μm) at ambient temperature to separate the particles from the receiving liquid wherein the particle size of entrapped tetrahydrocurcumin (tetrahydrocurcumin loaded poly (L-lactide) is in the range of about 80-100 nm.

The superior biological activity of tetrahydrocurcumin loaded poly (L-lactide) has been exemplified in the undersaid illustrative example.

### EXAMPLE 1—SUPEROXIDE SCAVENGING ACTIVITY OF TETRAHYDROCURCUMIN AND TETRAHYDROCURCUMIN LOADED POLY (L-LACTIDE)

The scavenging activity towards the superoxide radical (O2⁻) was measured as the inhibition of superoxide radical (O2⁻) generation. The method was performed by using alkaline DMSO method. Potassium superoxide and dry DMSO were allowed to stand in contact for 24 h and the solution was filtered immediately before use. The filtrate (200 μl) was added to 2.8 ml of an aqueous solution containing Nitro blue tetrazolium-NBT (56 nM), EDTA (10 nM) and potassium phosphate buffer (10 mM). tetrahydrocurcumin/ tetrahydrocurcumin loaded poly (L-lactide) 10, 100 and 500 nM concentrations were added and the absorbance was recorded at 560 nm against a control in which pure DMSO was added instead of alkaline DMSO (Henry et al, 1976). Ascorbic acid was used as standard. The experiment was repeated in triplicate and the percentage scavenging was calculated by using the formula:

Inhibition (%)=[(Control−Test)/Control]×100

Results: Tetrahydrocurcumin, tetrahydrocurcumin loaded poly (L-lactide) and TROLOX control showed concentration dependant scavenging of superoxide radical (O2⁻) radical. At 100 μM concentration the scavenging potential of tetrahydrocurcumin was 50.88%, while TROLOX was 47.80% and tetrahydrocurcumin loaded poly (L-lactide) was 76.94%.

DPPH Radical Scavenging Assay

To 1 ml of 1, 5 and 50 μM concentrations of tetrahydrocurcumin/tetrahydrocurcumin loaded poly (L-lactide), 1 ml solution of DPPH (0.1 mM) was added. An equal amount of methanol and DPPH served as control. After 20 min of incubation in the dark, absorbance was recorded at 517 nm and the experiment was performed in triplicate. Ascorbic acid was used as standard. The percentage scavenging was calculated using the same formula as given above.

At 50 μM concentration plain tetrahydrocurcumin exhibited % scavenging of 48.2*1.50. At the same concentration, tetrahydrocurcumin loaded poly (L-lactide) exhibited better scavenging activity of 64.48±1.75. TROLOX was least effective at 38.4*0.75.

Thus it was exemplified that tetrahydrocurcumin loaded poly (L-lactide) obtained by the process outlined in the instant invention has superior free radical scavenging activity. On similar lines, it can be expected that the other biological functions like anti-carcinogenic, anti-diabetic, anti-lipidogenic, anti-hypercholesterolemic, anti-microbial effects of tetrahydrocurcumin loaded poly (L-lactide) will be superior to that bulk tetrahydrocurcumin.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A process of nanoencapsulating curcuminoid metabolites like tetrahydrocurcumin, said process comprising steps of
a) Expanding a solution containing tetrahydrocurcumin (THC) 2% and poly(L-lactide) 2% in a mixture of ethanol and carbon dioxide (3:2 wt/wt) across a nozzle (50 μm diameter and L/D=4) into water with a pre-established constant pre-expansion temperature (Tpre) of 55-100 deg C and pre-expansion pressure (Ppre) of 210-330 bar,
b) Collecting the precipitates (nanoencapsulated tetrahydrocurcumin) as a suspension at about 10-15 cm from the tip of the nozzle; and
c) Filtering the suspension obtained in step b through vacuum suction through a nylon membrane (0.45 gm) at ambient temperature to separate the particles from the receiving liquid wherein the particle size of entrapped tetrahydrocurcumin (tetrahydrocurcumin loaded poly (L-lactide) is in the range of about 80-100 nm.

2. Use of tetrahydrocurcumin loaded poly (L-lactide) of claim 1 in a process of free radical scavenging (inhibition of superoxide radical (O₂—) generation) using natural curcuminoid metabolites to bring about the effect of enhanced scavenging activity when compared to bulk tetrahydrocurcumin wherein the process comprises steps of,
a) Allowing Potassium superoxide and dry DMSO to stand in contact for 24 h;
b) Filtering the solution of step 1 immediately before use;
c) Adding 200 μl of filtrate to 2.8 ml of an aqueous solution containing Nitro blue tetrazolium-NBT (56 μM), EDTA (10 μM) and potassium phosphate buffer (10 mM);
d) Adding tetrahydrocurcumin/tetrahydrocurcumin loaded poly (L-lactide) at 10, 100 and 500 μM concentrations to the solution of step c;
e) Recording the absorbance at 560 nm against a control in which pure DMSO was added instead of alkaline DMSO; and
f) Calculating the percentage scavenging using the formula: Inhibition (%)=[(Control−Test)/Control]×100

3. Use of tetrahydrocurcumin loaded poly (L-lactide) of claim 1 in a process of free radical scavenging (DPPH scavenging) using natural curcuminoid metabolites to bring about the effect of enhanced scavenging activity when compared to bulk tetrahydrocurcumin wherein the process comprises steps of,
a) Adding to 1 ml of 1, 5 and 50 μM concentrations of tetrahydrocurcumin/tetrahydrocurcumin loaded poly (L-lactide), 1 ml solution of DPPH (0.1 mM)
b) Incubating the solutions of step a for 20 mM in the dark;
c) Recording absorbance at 517 nm; and
d) Calculating the percentage scavenging using the formula: Inhibition (%)=[(Control−Test)/Control]×100

4. The use according to claims 2 and 3 wherein natural curcuminoid metabolites may be selected from the group comprising of tetrahydrodemethoxycurcumin loaded poly (L-lactide), tetrahydrobisdemethoxycurcumin loaded poly (L-lactide), Hexahydrocurcumin loaded poly (L-lactide) and octahydrocurcumin loaded poly (L-lactide).
